(19) United States
(12) Patent Application Publication
(10) Pub. No.: US 2025/0375561 A1
(43) Pub. Date: Dec. 11, 2025

(54) FLUID MONITORING SYSTEM AND METHOD

(71) Applicant: Analog Devices International Unlimited Company, Limerick (IE)

(72) Inventors: Youri PONOMAREV, Rotselaar (BE); Claire LEAHY, Listowel (IE); Liam RIORDAN, Patrickswell (IE)

(21) Appl. No.: 18/737,509

(22) Filed: Jun. 7, 2024

(51) Int. Cl. A61M 1/36

(52) U.S. Cl. CPC A61M 1/367; A61M 2205/3327

(57) ABSTRACT

A fluid monitoring system for determining a property of a test fluid comprises a main fluid flow path through which a test fluid can flow; a primary sensor set comprising a primary sensor configured to provide a primary measurement signal indicative of a first property of a test fluid flowing through the main fluid flow path; a secondary sensor set for use in calibrating the primary sensor set, the secondary sensor set comprising a secondary sensor configured to provide a secondary measurement signal indicative of the first property of the test fluid, wherein the system is configured such that the secondary sensor set can selectively interact with test fluid; and a control unit configured to: acquire a first measurement based on the primary measurement signal and acquire a second measurement based on the secondary measurement signal; compare the first measurement and the second measurement in a calibration operation to determine an adjustment for calibrating the primary sensor set based on the secondary sensor set; and determine a first property of the test fluid based on a primary measurement signal of the primary sensor and the adjustment.

### FIELD OF THE DISCLOSURE

This disclosure relates to a fluid monitoring system for determining a property of a test fluid and a method of determining a property of a test fluid.

### BACKGROUND

Continuous monitoring of properties or parameters of various fluids is used in many industries, including the monitoring of bodily fluids, such as blood. For example, monitoring of blood in extracorporeal blood circuits is important as the blood is removed from the body but is then reintroduced back into the body. Extracorporeal blood circuits are used, for example in the case of acute kidney injury (AKI), sepsis and invasive cardiac/pulmonary surgeries, where treatment and/or surgery can last for several hours or days. Other processes relying on continuous fluid flow also require continuous monitoring, such as in bioprocessing. There is therefore a desire for uninterrupted monitoring throughout the duration of these and other processes.

### SUMMARY OF THE DISCLOSURE

In one aspect, a fluid monitoring system for determining a property of a test fluid is provided, the fluid monitoring system comprising: a main fluid flow path through which a test fluid can flow; a primary sensor set comprising at least one primary sensor configured to provide a primary measurement signal indicative of a first property of a test fluid flowing through the main fluid flow path; a secondary sensor set for use in calibrating the primary sensor set, the secondary sensor set comprising a secondary sensor configured to provide a secondary measurement signal indicative of the first property of the test fluid, wherein the system is configured such that the secondary sensor set can selectively interact with test fluid; and a control unit configured to: acquire a first measurement based on the primary measurement signal of the at least one primary sensor and acquire a second measurement based on the secondary measurement signal of the secondary sensor; compare the first measurement and the second measurement in a calibration operation to determine an adjustment for calibrating the primary sensor set based on the secondary sensor set; and determine a first property of the test fluid based on (i) a primary measurement signal of at least one primary sensor of the primary sensor set from the first measurement and/or a further measurement and (ii) the adjustment.

In another aspect, an extracorporeal bodily fluid system comprising a circulatory fluid flow path is provided, the system comprising the fluid monitoring system according to any other aspect, wherein the main fluid flow path is a circulatory fluid flow path for a bodily fluid.

In one aspect, a method of determining a property of a test fluid provided in a main fluid flow path of a fluid monitoring system is provided, the method comprising: acquiring a first measurement based on a primary measurement signal from a primary sensor of a primary sensor set comprising at least one primary sensor, wherein the primary measurement signal is indicative of a first property of a test fluid flowing through a main fluid flow path; causing a secondary sensor of a secondary sensor set to interact with the test fluid and acquiring a second measurement based on a secondary measurement signal from a secondary sensor of a secondary sensor set, wherein the secondary measurement signal is indicative of the first property of the test fluid; comparing the first measurement and the second measurement in a calibration operation to determine an adjustment for calibrating the primary sensor set based on the secondary sensor set; and determining a first property of the test fluid based on (i) a primary measurement signal of at least one primary sensor of the primary sensor set from the first measurement and/or a further measurement and (ii) the adjustment.

In another aspect, a computer program is provided, comprising computer program code configured, when said computer program is run on one or more physical computing devices, to cause said one or more physical computing devices to implement the method disclosed.

In another aspect, one or more non-transitory computer readable media is provided, having a computer program stored thereon, the computer program comprising computer program code which is configured, when said computer program is run on one or more physical computing devices, to cause one or more physical computing devices to implement the method disclosed.

### BRIEF DESCRIPTION OF THE DRAWINGS

The present disclosure will now be described in more detail with reference to the accompanying drawings, which are not intended to be limiting:

FIG. 1 provides a schematic view of a fluid monitoring system according to an embodiment;

FIG. 2 provides a schematic view of a fluid monitoring system according to an embodiment;

FIG. 3 provides a schematic view of a fluid monitoring system according to an embodiment;

FIG. 4 provides a schematic view of a fluid system according to an embodiment;

FIG. 5 provides a schematic view of an extracorporeal bodily fluid system according to an embodiment; and

FIG. 6 provides a schematic view of a method according to an embodiment.

### DETAILED DESCRIPTION

In one aspect, a fluid monitoring system for determining a property of a test fluid comprises: a main fluid flow path through which a test fluid can flow; a primary sensor set comprising at least one primary sensor configured to provide a primary measurement signal indicative of a first property of a test fluid flowing through the main fluid flow path; a secondary sensor set for use in calibrating the primary sensor set, the secondary sensor set comprising a secondary sensor configured to provide a secondary measurement signal indicative of the first property of the test fluid, wherein the system is configured such that the secondary sensor set can selectively interact with test fluid; and a control unit configured to: acquire a first measurement based on the primary measurement signal of the at least one primary sensor and acquire a second measurement based on the secondary measurement signal of the secondary sensor; compare the first measurement and the second measurement in a calibration operation to determine an adjustment for calibrating the primary sensor set based on the secondary sensor set; and determine a first property of the test fluid based on (i) a primary measurement signal of at least one primary sensor of the primary sensor set from the first measurement and/or a further measurement and (ii) the adjustment.

The fluid monitoring system allows for a property of a test fluid to be measured within a system. This system further provides for calibration of a primary sensor within primary sensor set though the use of a secondary sensor within a secondary sensor set, where the secondary sensor set selectively interacts with the test fluid. This may contribute to an increase in the accuracy of the primary sensor set.

Specifically, the system may allow for a primary sensor set to be adjusted for improved accuracy using a secondary sensor set. The primary sensor set is configured to provide a primary measurement signal indicative of a first property of a test fluid flowing through the main fluid flow and, therefore, may be exposed continuously to the test fluid and/or may be used to carry out numerous measurements over time. This can lead to sensor drift and a general decrease in accuracy and precision over time. In contrast, the secondary sensor set can selectively interact with the test fluid allowing for the system to limit the exposure of the secondary sensor set to the test fluid, and hence may reduce these effects by limiting some of the mechanisms by which a decrease in accuracy and/or precision of sensors may occur. Accordingly, when the secondary sensor set is used to measure the first property, it is expected that this will provide a more accurate and precise measurement of the first property than a measurement from the primary sensor. The two measurements can then be compared, and the comparison can be used to correct the system so that the measurements carried out by the primary sensor are more accurate. For example, aligned to what a corresponding measurement from the secondary sensor would indicate. This can lead to improved sensor performance and may allow for extended use of a primary sensor.

Moreover, calibration in this way may provide significant operational benefits. Calibration of primary sensors using other methods may lead to interruptions and downtime or increased burden. For example, compared to an arrangement in which the primary sensor could be calibrated directly using a calibration fluid, which in turn may require halting the flow of the test fluid in the flow path and/or disengagement or replacement of the primary sensor from the primary fluid flow path, the method of the disclosure may allow a primary sensor set to continue operating without interruption or need of replacement during operation. This may forgo the need for fluid sampling and subsequent external laboratory testing to determine if the primary sensors are subject to drift and require replacement, which may be labour intensive and limit or prevent point-of-care applications. This can also be advantageous as the sensing process can be carried out over a longer time period, increasing sensitivity.

Further, the secondary sensors can be stored in calibration fluid between measurements, ensuring or improving accuracy and only removed when the secondary sensors are due to selectively interact with the test fluid. This has significant advantages. For example, extracorporeal bodily fluid systems will be connected to a patient for many hours, even days, and continuous automatic sampling and sensing is required without failure and, preferably, without interruption. In the context of bodily fluids, it will be appreciated that strict regulations on contamination and patient health mean that only regulator-approved fluids can be introduced into a patient. Embodiments provide a system where the secondary sensor can interact with these fluids (e.g. a calibrant) in a position where it is not selectively engaged with or interacting with the test fluid. Any such fluid can then be removed or flushed with a biocompatible fluid before interacting with the test fluid. Similarly, in other sensing environments, there are similar benefits where the test fluid integrity may need to be maintained. This may also mean that there is no restriction regarding the type and hazard levels of the calibrants. Cost effectiveness and ease of use may therefore be the improved. In such embodiments, the freedom regarding the calibration fluid may arise if the fluid from the secondary sensor set is not returned to the main fluid flow path.

There are several ways that the system is able to determine a first property of the test fluid based on a primary measurement signal of the primary sensor. Within the system, the determined first property is determined based on a primary measurement signal of the primary sensor and the adjustment.

Each measurement (i.e. the first measurement, second measurement or any further measurement) may comprise a read of the measurement signal caused by the control unit addressing or interrogating the sensor—i.e. the control unit obtaining the measurement signal. For example, electrochemical sensors may be used for example to convert a physical process into a digital signal corresponding to voltage amplitude for example. A transducer may be used in the conversion of physical signals related to ion concentration, temperature or light intensity to digital signals. The measurement may comprise obtaining these signals. Alternatively or additionally, the measurement may comprise an operation performed on the respective measurement signals. In other words, the first, second and/or further measurement may comprise converting the measurement signal into an intermediate value (before a corresponding property) and/or into the property to be measured. This can involve converting the raw data into a property, such as concentration. In such embodiments, the measurement may comprise obtaining a measurement signal from a corresponding sensor or the signal may otherwise be provided to the control unit. In some embodiments, an intermediate value may be determined between obtaining a primary or secondary measurement signal and acquiring a first or second measurement but, in other embodiments, the signal may be directly converted to or correlated with a property. Accordingly, the adjustment may be determined based on a comparison of the signal directly or any value derived from the signal.

The determination of ion concentration within an electrochemical signal may be dependent on diffusion rates within a fluid which may in turn depend upon temperature. As such, the calibration of a primary sensor may be achieved through the use of a plurality of sensors within the secondary sensor set, or through the use of a secondary sensor within the secondary set which does not measure the same parameter, but still allows for calibration of the primary sensor within the primary set (e.g. temperature and ion concentration).

In some embodiments, the at least one primary sensor of the primary sensor set may provide the primary measurement signal(s) (or the control unit may be configured to obtain measurement signals at intervals of) at an interval of: from 0.1 seconds to 5 hours, from 0.1 seconds to 1 hour, from 1 second to 30 minutes, from 0.1 seconds to 1 minute, from 0.1 seconds to 10 seconds, from 30 seconds to 1 hour, from 30 seconds to 30 minutes, from 30 seconds to 1 minute, from 1 minute to 1 hour or from 1 minute to 30 minutes. In some embodiments an individual sensor of the secondary sensor set (i.e. at least one secondary sensor) may provide the secondary measurement signal(s) (or the control unit may be configured to obtain measurement signals at intervals of) at an interval of: from 0.1 seconds to 5 hours, from 0.1 seconds to 1 hour, from 1 second to 30 minutes, from 0.1 seconds to 1 minute, from 0.1 seconds to 10 seconds, from 30 seconds to 1 hour, from 30 seconds to 30 minutes, from 30 seconds to 1 minute, from 1 minute to 1 hour or from 1 minute to 30 minutes.

In one aspect, a method of determining a property of a test fluid provided in a main fluid flow path of a fluid monitoring system comprises: acquiring a first measurement based on a primary measurement signal from a primary sensor of a primary sensor set comprising at least one primary sensor, wherein the primary measurement signal is indicative of a first property of a test fluid flowing through a main fluid flow path; causing a secondary sensor of a secondary sensor set to interact with the test fluid and acquiring a second measurement based on a secondary measurement signal from a secondary sensor of a secondary sensor set, wherein the secondary measurement signal is indicative of the first property of the test fluid; comparing the first measurement and the second measurement in a calibration operation to determine an adjustment for calibrating the primary sensor set based on the secondary sensor set; and determining a first property of the test fluid based on (i) a primary measurement signal of at least one primary sensor of the primary sensor set from the first measurement and/or a further measurement and (ii) the adjustment.

### Determining a Property of the Fluid

The methods and systems disclosed herein can be used to determine or measure a property of a test fluid. By test fluid, it is meant a fluid, such as a liquid or a gas, which is to be monitored. This may be a portion or the whole of the fluid. A flow of the fluid may be created within a flow path, such that the whole of fluid is monitored by passing through the main fluid path. For example, within a medical context, the fluid to be monitored may be a bodily fluid, such as blood. The continuous monitoring of a blood parameter is important within medical therapies within a critical patient care context. Examples of this include the monitoring of acute kidney injury, sepsis treatments and invasive cardiac or pulmonary surgeries.

Each of the primary and secondary sensors within the system within the respective primary set of sensors and the secondary set is configured to provide a measurement signal indicative of a property of the fluid. The sensors may comprise or be electrochemical sensors (potentiometric, amperometric, impedimetric), optical sensors (absorption, reflection, fluorescence), and/or acoustic sensors (photoacoustic, ultrasound). The property may be selected from an analyte characteristic, conductivity, temperature, or any other property of a relevant fluid. In embodiments, the analyte characteristic may include detection of the concentration of the analyte in the test fluid. For example, the property may be selected from: the concentration or presence of one or more of the following ions: Na⁺, K⁺, Ca²⁺, Mg²⁺, Cl⁻ and NH₄⁺; the concentration of [H⁺] ions, such that pH can be determined; metabolite concentrations, such as glucose, creatinine or lactate; the concentration of dissolved gases O₂, CO₂ or N₂ (such as measuring the partial pressure of the respective dissolved gas); the concentration or presence of certain biomarkers including cytokines, DNA and RNA. The sensors may further measure physical parameters of a fluid including conductivity and temperature. In embodiments, the system is configured for detection of at least one property (i.e., at least the first property) in a body fluid. In embodiments, this can be blood, urea or saliva.

The primary measurement signal indicative of a first property of a test fluid flowing through the main fluid path can represent the measurement signal which is obtained from or via the primary sensor or the signal which is processed. The control unit then receives the first measurement and adjusts it or a value derived from this, which could be an initial indication of the first property itself, (i.e. the measurement) to provide an accurate determination of the first property. The determination of the first property can be based on the primary measurement signal of the first measurement and/or determined based on a further measurement that is made. That is, in the step of determining the first property, the measurement signal to which the adjustment is directly or indirectly applied may be the same primary measurement signal on which was used in the calibration operation or may be a further primary signal measured, or a combination of these.

The determination of the first property of the test fluid may be based on the primary measurement signal indicative of a first property of the test fluid obtained in the first measurement and/or the first property of the test fluid may be based on a further measurement indicative of a first property of the sample. This further measurement may be taken from the same primary sensor, or where there are plural primary sensors in the primary sensor set, a different primary sensor within the primary set of sensors which provides the primary measurement signal indicative of a property, such as the first property.

In some embodiments, the control unit is configured such that the primary measurement signal used in determining the first property is the primary measurement signal of the first measurement, and wherein determining the first property comprises applying the adjustment to the first measurement. In some embodiments, the step of determining the first property is based on the primary measurement signal of the first measurement and comprises applying the adjustment to the first measurement. In these embodiments, the first property that is determined is based on the primary measurement signal and the adjustment is determined based on the secondary measurement signal and the same primary measurement signal. This may improve precision and/or accuracy as the measurement for which the adjustment is determined is the same measurement from which the first property is determined from based on the primary measurement and the adjustment.

In some embodiments, the control unit is configured such that the primary measurement signal used in determining the first property is a primary measurement signal obtained in a further measurement, and wherein the adjustment is used in obtaining the primary measurement signal in the further measurement. In some embodiments of the method, the step of determining the first property is based on a primary measurement signal obtained in a further measurement, and wherein the adjustment is used in obtaining the primary measurement signal in the further measurement.

By primary measurement signal obtained in a further measurement, it is meant that a measurement is in addition to the primary measurement signal taken by the primary sensor in the first measurement is taken. The additional measurement may be taken from the primary sensor at different point in time to the primary measurement signal of the first measurement.

Within these embodiments, the adjustment is determined based on the primary measurement signal of the first measurement and the determination of the first property is based on the further measurement.

In some embodiments the control unit is configured to obtain a plurality of measurements based on the primary measurement signal; and wherein the control unit is configured determine a first property of the test fluid for each of the plurality of measurements based on the primary measurement signal for each measurement and the adjustment. Through taking a plurality of measurements based on the primary measurement signal of the primary sensor, information may be obtained relating to a first property of the test fluid over time and/or at different points in the flow. Moreover, the application of the adjustment to each of these allows for a single calibration point to be determined and used to correct or adjust other measurements within a particular time window, which can be, for example, an operation performed on previous measurements (such as since the start of the measurement of the first property or since the last calibration operation was performed) and/or on future measurements (such as until the measurements are no longer taken or until the next calibration operation is performed). The ratio of measurements to calibration operations can be optimised to balance the need for the secondary sensor set to be well-calibrated and the need for monitoring sensor drift or drop in performance of the primary sensor set.

Measurement of the first property over time can be useful in situations where fluid is subject to continuous monitoring. Within the context of fluid monitoring, the determination of a property of a sample fluid may be uninterrupted for the duration for which monitoring is desired. As such, the use of a secondary sensor set which serves the purpose of calibrating the primary sensor set may allow for calibration without having to stop the flow of fluid through the primary sensor set in order to calibrate the primary sensor set.

In some embodiments, the test fluid to be monitored may be a bodily fluid, such as blood. The continuous monitoring of a blood parameter is important within medical therapies critical to patient care. Examples of this include the monitoring of acute kidney injury, sepsis treatments and invasive cardiac or pulmonary surgeries. The system may allow for a primary sensor set that acquires a first measurement to be adjusted for improved accuracy using a secondary sensor set that acquires a second measurement. This may allow a primary sensor set to continue operating without need of replacement during operation. If the secondary sensor set were not present, the calibration of the primary sensor set may pose issues to an operator of the system as it is generally not possible to re-calibrate sensors in line during operation. For example, because calibrant for the primary sensor set may be incompatible with or contaminate a patient's blood in the main fluid flow. The use of a secondary sensor set therefore may avoid such contamination and may thus improve the safety of patients.

In other embodiments, the system may, for example, be used in monitoring solutions such as monitoring which requires stable sensor performance over a significant amount of time. An example of such use would be within the bioprocessing field.

### Sensors and Sensor Sets

The primary sensor set comprises at least one primary sensor. The primary sensor set can comprise more than one sensor, each sensor configured to provide a primary measurement signal indicative of to a property (which may be the first property or another property) of a test fluid flowing through the main fluid flow path and, in some embodiments, more than one type of sensor. The primary sensor may in some embodiments contain more than one type of sensor for measuring a particular property or parameter. Examples include the use of different electrochemical sensors for measuring the concentration of a particular analyte, such as an ion. The primary sensor is configured to provide a primary measurement signal indicative of a first property of a test fluid flowing through the main fluid flow path. In some embodiments, at least one primary sensor in the primary sensor set may be provided in the primary fluid flow path (such as an electrode provided in the fluid flow path). In additional or alternative embodiments, at least one primary sensor may be external to the primary fluid flow path but be arranged so as to be able to interrogate the test fluid within the fluid flow path (such as an optical sensor).

The secondary sensor set comprises at least one secondary sensor. The secondary sensor set can comprise more than one sensor, each sensor configured to provide a primary measurement signal indicative of a property (which may be the first property or another property) of a test fluid flowing through the main fluid flow path and, in some embodiments, more than one type of sensor. The secondary sensor in some embodiments may contain more than one type of sensor for measuring a particular property or parameter. Examples include different electrochemical sensors for measuring the concentration of an ion.

The secondary sensor is configured to provide a secondary-measurement signal indicative of the first property of the test fluid. The system is configured such that the secondary sensor set can selectively interact with test fluid. By this it is meant that the system is configured so that the test fluid or a portion thereof can selectively be provided to the secondary sensor set (such as intermittently). In this way, there is not continuous exposure of the secondary sensor to the test fluid and sensor drift and/or contamination of the secondary sensor set can be reduced or avoided. In other words, the system is configured so that the secondary sensor set is exposed to/interacts with the test fluid for a shorter period of time than the primary sensor set. The selective interaction can be provided by moving the sensor set into or out of engagement with the test fluid. In some embodiments, a portion of the test fluid may be diverted from the main fluid flow path to the secondary sensor set, which may be provided in a separate fluid flow path. The control unit may be configured to control the selective interaction, for example so as to divert a portion of the test flow to the secondary sensor set.

The primary sensor and the secondary sensor are both configured to provide a measurement signal indicative of a first property (i.e., the same property). They may be the same type of sensor, such as a sensor having the same modality and mode of operation, or, in some embodiments, the modality of the primary sensor and secondary sensor are different. Where there are plural primary sensors and plural secondary sensors in the primary and secondary sensor sets, respectively, each of the primary sensors for detecting a particular property may have the same modality as a corresponding sensor for detecting the that particular property.

Sensing modality refers to the means by which a measurement signal indicative of a property of the test fluid is determined. A first and second sensing modality may be the same, for example two identical electrochemical sensors for measuring ion concentration, with one sensor set containing each. The modality refers to the means of sensing, so the two electrochemical sensors may not measure the same property.

In some embodiments, the primary sensor has a first sensing modality and the secondary sensor has a second sensing modality. In some embodiments, the method further comprises having a first sensing modality and providing a secondary sensor having a second sensing modality.

In some embodiments, a first and second sensing modality refers to the use of one type of sensor in the primary sensor set and at least one different type of sensor in the secondary sensor set but both are used to determine the first property. Any sensor not relying on the same underlying reaction or measuring means is of a different modality. For example, a first sensor measuring an ion concentration through the use of one electrochemical reaction and a second sensor measuring the concentration of the same ion species through the use of a second, different reaction would qualify as different types of sensors i.e. having different modality. Types of sensor modality include electrochemical, optical and acoustic. Types of sensors can further include electrochemical potentiometric, electrochemical amperometric, electrochemical impedimetric, optical absorption, optical reflection, optical fluorescence, acoustic photoacoustic and acoustic ultrasound. The skilled reader will appreciate that many other sensors are available for sensing. The use of different sensing modalities between the primary and secondary sensors may allow for limitations of one sensor type (e.g. optical sensors, which can be prone to interference and drift) can be corrected for using a different modality (such as electrochemical sensors) which may be more precise and accurate, but which may not be suited to extensive exposure to a test fluid and accordingly are not suited to the level of exposure that an optical sensor may be able to tolerate. Thus, different problems which may be accounted for by having sensors of different modalities and of different forms (i.e., optical, electrochemical and acoustic).

The use of different sensing modalities within each of the primary and secondary sensors may also allow for confirmatory measurements to be made. For example, if an ion species concentration is being measured by a sensor (A) in the electrode in the secondary sensor set, this can calibrate a sensor of the same modality (A-A) in the primary sensor set. This setup may be used in combination with a sensor of a different modality (B) within the primary sensor set. This sensor of a different modality (B) in the primary set may be calibrated separately using a corresponding sensor modality (B) in the secondary set. Both sets of sensors (A,B) may have different modalities meaning that a user can obtain two independent results relating to a single parameter. One potential advantage of this is that different limitations apply to different sensors which may be accounted for by having sensors of different modalities and of different forms (i.e., optical, electrochemical and acoustic).

In some embodiments, a third sensor set may be provided comprising at least one third sensor configured to provide a third sensor signal. The calibration operation may comprise obtaining a third measurement signal and basing the adjustment in part on the third measurement signal. This may be separately to or in addition to the second measurement signal. This may be advantageous as different sensors may not be prone to the same types of error or drift. Furthermore, this setup may be advantageous since the one sensor set may be replaced while the other sensor set is still operative. This may be advantageous since it allows for calibrated measurements of the primary sensor set to be made without any period in which the measurements cannot be calibrated against any calibration unit sensor set. In some embodiments, the sensors of the secondary sensor set may be of a first modality and the sensors of the third sensor.

In some embodiments, the primary sensor comprises an optical sensor and the secondary sensor comprises an electrode for electrochemically sensing the first property.

In some embodiments, the primary sensor set comprises at least one further primary sensor configured to provide a further primary measurement signal indicative of a further property of a test fluid flowing through the main fluid flow path; and wherein the secondary sensor set further comprises at least one further secondary sensor, each further secondary sensor being configured to provide a further secondary measurement signal indicative of a corresponding further property of the test fluid. By taking a plurality of measurements based on the primary measurement signal, the control unit is able to determine multiple properties within the system and, further, calibrate those sensors. In a fluid treatment context, many different parameters need to be measured and calibrated, therefore the use of more than one primary sensor and more than one secondary sensor facilitates this. This may allow for a clinician, in a medical context to obtain a more complete understanding of a patient's blood system. This may lead to increased efficiency in responding to changes within a patient's blood system.

As set out above, in some embodiments, there may be a plurality of primary sensors in the first sensor set and a plurality of secondary sensors in the secondary sensor set. In such embodiments, there may be a corresponding primary and secondary sensor such that each primary sensor configured to detect a particular property (i.e., provide a measurement signal indicative of that particular property) has a corresponding secondary sensor configured to detect that particular property.

In some embodiments where there are further primary sensors, the further primary sensors may be a sensor of the same modality or of a different modality. Furthermore, both the primary sensor and further primary sensor may be identical. The secondary sensor and further secondary sensor may be identical. This may serve the function of providing back-up secondary information through additional sensors. The primary and secondary further sensors may operate differently over different parameter ranges to the primary and secondary sensors. In this manner, the signal response vs concentration curves may be different for the sensors and further sensors. In some embodiments, the sensors can selectively be turned on and off based on a measured value indicated by the system. For example, a first electrochemical sensor may be particularly sensitive to changes for an ion species concentration within a given order of magnitude, whereas a second electrochemical may be particularly sensitive to changes within an ion species within a second ion species within a different order of magnitude.

### Adjustment and Calibration

By “adjustment”, it is meant that a correction is determined which takes account of the comparison of the primary sensor set and secondary sensor set. The adjustment may be a value (such as for correcting the measurement signal or property value) or an operator, such as an equation. The adjustment may be fixed or may be variable depending on the value to which it is applied. For example, the adjustment may be different depending on the value of the measurement, such as the concentration. The adjustment may take place via a single linear addition or subtraction to take account for an offset between the two values. Alternatively, the adjustment may result in some adjustment factor which the primary measurement is multiplied by. More complex processing may occur at this stage. For example, in some embodiments, a formula for determining the calibration operation to be performed may be determined through the use of one or more measurements in the second set of sensors. Furthermore, changes over time may be identified within the system and factored into the calibration operation.

There are several means by which the adjustment within the system. This may, for example, if electrochemical sensors are used within the primary and/or secondary sensor set, correspond to adjusting the voltage or current reading that is being read from a sensor. Alternatively or additionally, the adjustment may take place on an intermediate processing value derived from the measurement signal, where such processing takes place. Alternatively or additionally, the adjustment may take place after the property has been determined so as to correct this initial property determination (which is subject to any sensor inaccuracy) and correct it to provide an accurate, final property determination.

Where there are plural primary sensors, particularly plural primary sensors configured to determine measurement signals of different properties or using different modalities, the adjustment may comprise a separate adjustment component for each of the plural primary sensors. Each adjustment component may be as defined above for the adjustment and each may be determined based on a corresponding secondary sensor configured to provide a measurement signal for a corresponding property.

The adjustment for the signal is determined by comparing the first and second measurements in a calibration operation. By calibration operation, it is meant that an operation or a series of operations are carried out on the measurements (e.g., the signals or a derivation from the signals) to determine an adjustment to be made.

The secondary sensor set provides secondary sensor(s) which selectively interact with the test fluid and hence may be at less at risk of sensor drift. In some embodiments, the system may be configured to calibrate the secondary sensor set prior to acquiring the second measurement and carrying out the calibration operation. That is, the secondary sensor(s) may be pre-calibrated sensor before the test fluid is provided to the secondary sensor set (such as received into the secondary flow path) and the secondary sensor set takes one or more measurements through the use of the one or more secondary sensors.

In some embodiments, the system comprises a calibration fluid reservoir for receiving a calibration fluid, wherein the system is configured so that the calibration fluid reservoir is in selective fluid communication with secondary sensor set so as to selectively provide calibration fluid to the secondary sensor set. In some embodiments, a calibration fluid may be provided within the reservoir. The system may be configured to provide calibration fluid from the fluid reservoir to the second sensor unit, for example the reservoir may be fluidly connected to the secondary fluid flow path (where present). The calibration fluid is a fluid provided for ensuring that the secondary sensors are accurate and do not drift during use. When not carrying out measurements, the secondary sensors can be immersed in the calibration fluid. Then, before a measurement of the test fluid is carried out, the calibration fluid can be removed (and optionally the secondary sensor set may be washed or flushed) and the measurement with the test fluid carried out. The control unit may be configured to control this process, such that it is configured to dispense calibration fluid to the secondary sensor unit and, prior to acquiring the secondary measurement signal for the calibration operation, is configured to cause the removal of the calibration fluid from the secondary fluid sensor and, subsequently, cause the test fluid to be provided to the secondary sensor set.

The calibration fluid being in selective communication with the secondary sensor set, and therefore not requiring the primary sensor set to come into contact with the calibration fluid, may have the advantage that there is no restriction regarding the type and hazard levels of the calibrants. Cost effectiveness and ease of use may therefore be improved. In such embodiments, freedom regarding the calibration fluid choice may arise if the fluid from the secondary sensor set is not returned to the main fluid flow path.

Although in the above embodiments, there is a calibration fluid reservoir, it will be appreciated that the secondary sensors may otherwise be calibrated, such as by application of an external calibration fluid.

In some embodiment, the system may further comprise a secondary calibration fluid reservoir for receiving a second calibration fluid (and in some embodiments, may further comprise the second calibration fluid) and with selective fluid communication with the secondary sensor set. Provision of a secondary calibration fluid which may be utilised by the secondary sensor set is advantageous as it may allow for selection of an appropriate calibration fluid based on the conditions, for example based on the measured properties of the fluid. The control unit may determine which calibration fluid should be used based on a measurement and dispense one of the calibration fluids to the secondary sensor set accordingly.

In some embodiments, the method may comprise, prior to acquiring the second measurement, calibrating the secondary sensor of the secondary sensor set. This may comprise providing a calibration fluid to the secondary sensor set, for example as detailed above.

The calibration fluid is a fluid provided for ensuring that the secondary sensors are accurate and do not drift during use. The calibration fluid may comprise known ion, metabolite, dissolved gas, biomarker concentrations as well as further known physical properties such as conductivity and temperature. Examples of such fluids include an aqueous solution of 3.1 mM KCl, 139 mM NaCl, 1.165 mM CaCl2, 10 mM HEPES and NaOH added (3-4 mM) to adjust to pH 7, where mM refers to millimoles per litre or 10⁻³ mol/L; Aqueous Lactated Ringer solution; and a physiological saline solution.

### Flow Path

In some embodiments, the system further comprises a secondary fluid flow path arranged to selectively receive a portion of the test fluid from the main fluid flow path, wherein the secondary sensor is configured to detect the first property of the test fluid when provided in the second fluid flow path.

In some embodiments of the method, further comprises providing a secondary fluid flow path arranged to selectively receive a portion of the test fluid from the main fluid flow path, wherein causing a secondary sensor of the secondary sensor set to interact with the test fluid comprises providing a portion of the test fluid from the main fluid flow path into the secondary fluid flow path; and acquiring the second measurement based on a secondary measurement signal from the test fluid when the test fluid is provided in the second fluid flow path.

The second fluid flow path being arranged to selectively receive a portion of the test fluid from the main fluid path may allow the second fluid to selectively or intermittently receive the test fluid from the main fluid path. Accordingly, the main and second fluid flow paths can be fluidly connected or fluidly connectable. In some embodiments, a valve may be provided to control flow of test fluid from the main flow path to the secondary fluid flow path. The control unit may be configured to control the valve to provide selective interaction of the secondary sensor set with the test fluid.

The test fluid may be discarded or otherwise retained outside of the main flow path and not returned to the source after measurement using the secondary sensor set. For example, where the test fluid is provided to a secondary fluid flow path, the secondary fluid flow path may be fluidly connected to a waste reservoir or to an outlet (from the system).

The selective interaction of the secondary sensor set may be controlled by the control unit based on a set time, such as set intervals or set numbers of primary measurements. In some embodiments, the times at which the second fluid flow path receives that test fluid from the main fluid path are regular time intervals. In some embodiments, the times at which the second fluid flow path receives the test fluid from the main fluid path are variable and may depend upon the adjustment that is carried out. The adjustment can be applied to the previous or future measurements of the primary sensor set during those intervals (i.e., the measurements adjacent the calibration operation).

In some embodiments, the selective receiving of a portion of the test fluid from the main fluid flow path corresponds to the times at which a measurement will be provided by a sensor of the secondary sensor set (or the times that the control unit may be configured to obtain measurement signals). In these embodiments, the secondary sensor set may receive fluid at different time intervals separated by a time within one of the following time ranges: from 0.1 seconds to 5 hours, from 0.1 seconds to 1 hour, from 1 second to 30 minutes, from 0.1 seconds to 1 minute, from 0.1 seconds to 10 seconds, from 30 seconds to 1 hour, from 30 seconds to 30 minutes, from 30 seconds to 1 minute, from 1 minute to 1 hour or from 1 minute to 30 minutes.

The secondary fluid flow path, where present, can be connected to the main fluid flow path at any point along the length of the main fluid flow path. In some embodiments, this may be where the second fluid flow path is fluidly connected to the main fluid flow path at the point the where the primary sensors set is provided. This may contribute to the secondary sensor set being able to more reliably calibrate the primary sensor set as the environment of the secondary sensor set will more closely reflect that of the primary sensor set. For example, the temperature or flow rate of the fluid may vary throughout its flow in the system, but having the secondary fluid flow path and secondary sensor set extracting fluid form the region at or adjacent the primary sensor set may contribute to closer testing conditions. For electrochemical sensors, the rate of diffusion of species within a fluid is temperature dependent. The rate of diffusion is an important factor in concentration measurements for electrochemical sensors as it determines the number of species reaching the sensor. Accordingly, consistency of temperature between the primary and secondary sensor set may contribute to improved reliability of the adjustments and therefore increased accuracy in properties which are determined.

In an aspect, an extracorporeal bodily fluid system comprising a circulatory fluid flow path, comprising the fluid monitoring system, wherein the main fluid flow path is a circulatory fluid flow path for a bodily fluid. By circulatory fluid flow path in extracorporeal bodily fluid sensing system, it is meant a fluid flow path which extracts a bodily fluid from the patient and then returns the fluid to the patient (e.g. in a circulatory manner). An extracorporeal bodily fluid system may also use a fluid flow path which is used to extract a bodily fluid from the patient and returns it to another. The bodily fluid may be treated or modified during circulation. The circulatory fluid flow path may be comprised of several flow paths, in parallel and/or series, and may include passing the bodily fluid through treatment devices or systems. Examples of systems incorporating such circulatory fluid flow paths include dialysis or extracorporeal membrane oxygenation (ECMO) systems. In embodiments, there is provided an extracorporeal bodily fluid sensing system comprising a fluidic device as disclosed herein and a circulatory fluid flow path in fluid communication with the fluidic device.

In some embodiments, the bodily fluid is blood. Examples of systems extracting and returning blood include dialysis (haemodialysis) and autotransfusion, cardiopulmonary bypass (heart-lung machine) or extracorporeal membrane oxygenation (ECMO) device. Blood being returned to a patient is subject to stringent requirements and should not be compromised, for example by contamination with a cleaning or calibration fluid.

The use of an extracorporeal bodily fluid system facilitates the testing of bodily fluid. In some embodiments, this involves the testing of blood. Within a clinical environment, this may have the advantage of allowing a clinician to obtain real-time data about one or more parameters within a patient's blood. For example, the balance of Na⁺ and K⁺ is of particular medical significance, therefore this ratio can be monitored by a clinician. Furthermore, other concentrations may be monitored such as blood glucose levels. It will be appreciated that many different sensors may be used to monitor a variety of different physical parameters. The system may have the advantage that the primary sensor set does not need to be taken out in order to be calibrated and can be calibrated externally using a secondary sensor set. This may have the advantage of allowing for continuous fluid flow through the primary sensor set within the main fluid flow path.

Within an extracorporeal bodily fluid system, in some embodiments, the control unit operates to determine a first property of the test fluid either after each measurement is taken by the primary set of sensors, whilst other measurements are occurring and/or after all measurements have occurred. This may enable clinicians to both identify changes in real-time and also observe changes within a patient over time. In some embodiments, historical trends within a patient's bloodstream may therefore be monitored.

In some embodiments, the method further comprises providing an extracorporeal bodily fluid system comprising a circulatory fluid flow path for a bodily fluid defining the main fluid flow path; and providing a bodily fluid to the circulatory fluid flow path, wherein the bodily fluid is the test fluid.

In some embodiments, the method may comprise providing a flow of test fluid over the primary sensor set. This may be a continuous flow of test fluid through the main fluid flow path for a period of time, such as at least 1 minute, at least 10 minutes, at least 1 hour and may be up to 48 hours, for example.

### System and Method

The system may be configured to perform any of the method steps disclosed herein. Moreover, any of the embodiments set out herein with respect to the method apply equally to the system, and any of the embodiments set out herein with respect to the system apply equally to the method. In embodiments, the control unit of the system may be configured to carry out any of the method steps set out herein.

The control unit may be or comprise one or more processors or controllers (provided locally, remotely and/or distributed across a network) and may be implemented in any suitable manner, with software and/or hardware, to perform the various functions required. One or all of the units may, for example, employ one or more microprocessors programmed using software (for example, microcode) to perform the required functions. Examples of processor components that may be employed in various embodiments include, but are not limited to, conventional microprocessors, application specific integrated circuits (ASICs), and field-programmable gate arrays (FPGAs). In various implementations, the control unit may be associated with one or more non-transitory storage media such as volatile and non-volatile computer memory such as RAM, PROM, EPROM, and EEPROM. The non-transitory storage media may be encoded with one or more programs that, when executed on one or more processors and/or controllers, perform the required functions. Various storage media may be fixed within a processor or controller or may be transportable, such that the one or more programs stored thereon can be loaded into the control unit.

In some non-limiting examples, the system includes a user interface, such as a display, for communicating the measurement signal, measurement or property determined by the control unit. Alternatively, or additionally, the system may include a communications interface device, such as a wireless transmitter, configured to transmit the measurement signal, measurement or property to an external device, such as a personal computer, tablet, smartphone, remote server, etc.

In an aspect, a computer program is provided, comprising computer program code configured, when said computer program is run on one or more physical computing devices, to cause said one or more physical computing devices to implement the method disclosed.

In an aspect, one or more non-transitory computer readable media having a computer program stored thereon is provided, the computer program comprising computer program code which is configured, when said computer program is run on one or more physical computing devices, to cause one or more physical computing devices to implement the method disclosed.

The system may comprise component parts. In some embodiments, the system comprises (i) a sensor assembly comprising at least a part of the main fluid flow path and the primary sensor set, (ii) a calibration unit comprising the secondary sensor set and at least a part of the secondary fluid flow path, and (iii) the control unit, which may optionally be a part of one or more of the sensor assembly and calibration unit. In some embodiments, the fluid monitoring system further comprises a calibration unit, the calibration unit comprising the secondary sensor set and the second fluid flow path, wherein the calibration unit is releasably engageable with the main fluid flow path. The system may therefore comprise a calibration unit which incorporates the secondary sensor set and the second fluid flow path, which can be disengaged from the part of the system incorporating the primary sensor set. This allows for replacement of the secondary sensor set, if required, without interruption to the measurements carried out by the primary sensor set.

### Specific Embodiments

Fluid monitoring systems are used to determine a property of a test fluid. Such systems can enable real-time monitoring or one or more parameters such that decisions can be made by an operator based on one or more measurements. Calibration operations within a fluid system can enable a greater level of precision, accuracy and reliability for measured properties of a fluid.

Within the figures, the direction of fluid flow is indicated by the arrows contained within the elements of the system.

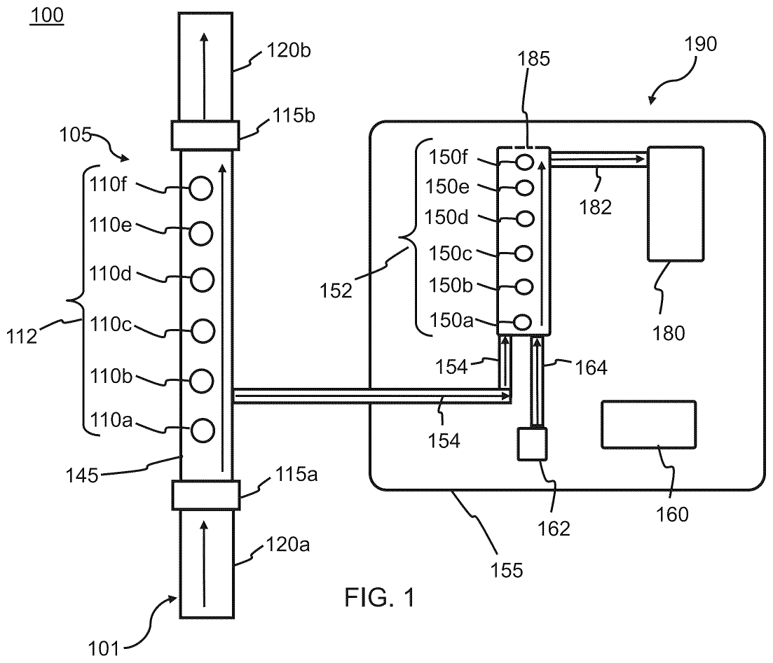

FIG. 1 schematically depicts a system for determining a property of a test fluid according to an embodiment. Specifically, FIG. 1 depicts a fluid monitoring system 100 for determining a property of the test fluid, comprising a fluid circulation device 101, a sensor assembly 105 and a calibration unit 190.

The sensor assembly 105 comprises a primary sensor set 112 which, in this embodiment, comprises six primary sensors 110a-f. Each of the primary sensors 110a-f is configured to provide a measurement signal corresponding to a different property of test fluid. The sensor assembly 105 also comprises an elongate primary sensor housing 145 in which the primary sensors 110a-110f are located and arranged in a row.

The fluid circulation device 101 is only partially depicted and is arranged such that test fluid can be circulated around a circulatory fluid flow path (not depicted in full), which circulatory fluid flow path defines a main fluid flow path 120. In this embodiment, the fluid circulation device 101 comprises a first conduit 120a which connects to an inlet of the primary sensor housing 145 via a connector 115a to provide fluid to the primary sensor housing 145. The fluid circulation device 101 also comprises a second conduit 120a which connects to an outlet of the primary sensor housing 145 via a connector 115b and receives test fluid from the primary sensor housing 145. Accordingly, together, the first conduit 120a, primary sensor housing 145 and second conduit 120b define the main fluid flow path 120 through which test fluid can continuously flow. As test fluid passes through the main fluid flow path 120, it will pass through the primary sensor housing 145 and over the primary sensors 110a-f so that these can interact with the test fluid. The connectors 115a, 115b facilitate that attachment of the primary sensor housing 145 containing the primary sensors 110a-f. Although not depicted, it will be appreciated that the first conduit and second conduit 120a, 120b are fluidly connected and there may be further components within this circulatory fluid flow path.

The calibration unit 190 comprises a calibration unit housing 155 which is releasably engageable with the fluid circulation device 101 and in which the components of the calibration unit 190 are located.

The calibration unit 190 comprises secondary sensor set 152 in this embodiment in this embodiment comprises six secondary sensors 150a-f. Each of the secondary sensors 150a-f is configured to provide a measurement signal corresponding to a different property of test fluid. The calibration unit 190 also comprises a secondary sensor conduit 185 in which the secondary sensors 150a-f are located and arranged in a row.

The calibration unit 190 is fluidly connected to the main fluid flow path 120 by a conduit which provides a secondary fluid flow path 154. In particular, the secondary fluid flow path 154 extends from an opening in the primary sensor housing 145 to an inlet of the secondary sensor conduit 185 to fluidly connect the main fluid flow path 120 to the secondary sensor set 152. A valve (not depicted) controls the flow of test fluid from the main fluid flow path 120 into the secondary fluid flow path 152. It will be appreciated that, although depicted as the second fluid flow path 154 extending from the primary sensor housing 145 adjacent the primary sensors, it could also extend from another part of the main fluid flow pathway 120. However, it is thought that the depicted configuration may contribute to the secondary sensor set 152 being able to more reliably contribute to the calibration of the primary sensor set 112. This is because the environment of the secondary sensor set 152 will more closely reflect that of the primary sensor set 112. For example, the temperature or flow rate of the fluid may vary throughout its flow in the system but having the secondary fluid flow path 154 and secondary sensor set 152 being relatively close to the primary sensor set 112 and sampling test fluid from the same portion of the test fluid may contribute to closer testing conditions. For electrochemical sensors, the rate of diffusion of species within a fluid is temperature dependent. The rate of diffusion is an important factor in concentration measurements for electrochemical sensors as it determines the number of species reaching the sensor. Accordingly, consistency of temperature between the primary 112 and secondary sensor set 152 may contribute to improved reliability of the adjustments and therefore increased accuracy in properties which are determined.

Each of the primary sensors 110a-f and secondary sensors 150a-f within the system 100 is configured to provide a measurement signal indicative of a particular property of the test fluid. Example sensor types include Electrochemical (potentiometric, amperometric, impedimetric), optical (absorption, reflection, fluorescence), and acoustic (photoacoustic, ultrasound).

The calibration unit 190 further comprises a waste conduit 182 which extends from an outlet of the secondary sensor conduit 185 to a waste chamber 180 so that fluid passing through the secondary sensor conduit 185 and the secondary sensor set 152 can be disposed of. A further valve may be provided at the outlet of the secondary sensor conduit 185 to control release of fluid from the secondary sensor conduit 185. This arrangement where fluid that has passed through the secondary sensor set 152 is not returned to the main fluid flow path 120 may further contribute to improved accuracy and helps to avoid or reduce risk of contamination of the test fluid in the main fluid flow path 120. This is because there may be no need for the calibrant to be biocompatible or non-toxic as there may be little to no risk of contamination of the test fluid in the main fluid flow path 120 will return to the source (e.g. a body). In such a setup the calibration fluid reservoir 162 may be filled with calibrants that otherwise would not have been selected due to their potential for infiltrating the system and causing hazards. This may be particularly advantageous within a medical context where blood contamination may pose a serious risk to patient safety.

The calibration unit 190 further comprises a calibration fluid reservoir 162 comprising a calibration fluid that can be used to calibrate the secondary sensor set. The calibration fluid may comprise a fluid with known ion, metabolite, dissolved gas, biomarker concentrations as well as further known physical properties such as conductivity and temperature. These values may be known from testing that has been carried out prior to the calibration fluid reservoir 162 receiving the calibration fluid. The calibration fluid is selected to contain known concentrations and/or physical properties that are selected such that they are within the ranges expected to be measured by the primary sensors 110a-f and secondary sensors 150a-f.

The calibration fluid reservoir 162 is connected to the inlet of the secondary sensor conduit 185 via a calibration flow path 164. The fluid communication is selective via a valve (not shown) so as to selectively provide calibration fluid to the secondary sensor set 152.

The system further comprises a control unit 160 configured to control operation of the various parts of the system. In this embodiment, control unit 160 is a processor provided in the calibration unit 190, but it will be appreciated that this could implemented in other ways, such as using plural processors or controllers provided locally or across a network. The control unit 160 in this embodiment includes a signal processing unit which can obtain a measurement signal from each of the primary sensors 110a-f and secondary sensors 150a-f and subsequently determine a property of a fluid (e.g., the test fluid) provided to the respect sensor based on the measurement signal. The control unit 160 further controls operation of the valve provided between the main fluid flow path 120 and the secondary fluid flow path 154 so as to control flow of test fluid from the main fluid flow path 120 to the secondary fluid flow path 154, and thus the secondary sensors 150a-f. The control unit 160 also controls the selective flow of fluid from the calibration fluid reservoir 162 to the secondary sensor conduit 185 and further from the secondary sensor conduit 185 to the waste chamber 180, which in this embodiment comprises control of the respective valves.

In use, the system 100 operates as a continuous fluid system whereby test fluid is continuously moving through the main fluid flow path 120. At regular intervals, the control unit 160 obtains a first measurement (or a plurality of first measurements) by taking a measurement signal from each of the primary sensors 110a-f corresponding to the property measured by the respective primary sensor 110a-f. The control unit 160 can use these measurement signals to calculate the various properties and report these to a user, for example by transmitting the result to an external device and/or by displaying the results on a display (not shown). However, during the continuous flow, the primary sensors 110a-f are continuously exposed to the test fluid and may be subject to sensor drift. This can lead to inaccurate and/or imprecise measurements and sensor failures.

Accordingly, the control unit 160 and system 100 are further configured to calibrate the primary sensors 110a-f so that the measurements are as accurate as possible. This is achieved by performing a calibration operation using the secondary sensor set 152, the secondary sensors 150a-f of which are not subject to the same prolonged exposure to the test fluid and, instead, can be in contact for extended periods of time with the calibration fluid and thus provide a stable and accurate reading for the respective property. This is achieved using input (i.e., a measurement) from both the primary sensors 110a-f and the secondary sensors 150a-f. The input from the primary sensors 110a-f may be the measurements(s) taken above or a specific measurement for the purposes of calibration but will be referred to herein as a “first measurement”. The first measurement used in the calibration may be the raw measurement signal, the final property value or any intermediate value or information derived therefrom.

If present, the control unit 160 may first remove the calibration fluid from the secondary sensor conduit 185. In this embodiment, this is achieved by the control unit 160 operating the valve at the waste conduit 182 to open it and allow the calibration fluid to drain and, if open, closing the valve between the calibration fluid reservoir 162 and the secondary sensor conduit 185. Although not depicted, a washing step may be used here. This could be using a flushing or cleaning solution. Alternatively or additionally, the test fluid could be used in excess to rinse the secondary sensor conduit 185.

To obtain a measurement from the secondary sensors 150a-f (herein the “second measurement”), the control unit 160 can then operates the valve between the main fluid flow path 120 and the secondary fluid flow path 154. This provides selective interaction of the secondary sensor set 152 with the test fluid. In this configuration, a secondary measurement signal is provided by each of the secondary sensors 150a-f corresponding to the respective property they are measuring. The control unit 160 acquires the first measurement signal from each of these sensors.

The control unit 160 then compares the first measurement and second measurement in a calibration operation to determine an adjustment for calibrating each of the primary sensors 110a-f. The adjustment is a correction which is determined which takes account of the comparison of the primary sensor set and secondary sensor set. The adjustment may be a value (such as for correcting the measurement signal or property value) or an operator, such as an equation. The adjustment may be fixed or may be variable depending on the value to which it is applied. For example, the adjustment may be different depending on the value of the measurement, such as the concentration. In this embodiment, each of the primary sensors 110a-f determines a different property of the test fluid and the secondary sensors 150a-f each determine one of these properties such that each primary sensor 110a-f has a corresponding secondary sensor 150a-f determining the same property, thereby providing a sensor pair. The adjustment is determined for each primary sensor 110a-f and secondary sensor 150a-f sensor pair so that there is an adjustment for each property that is to be determined.

The control unit 160 can then determine the property. This is based on (i) a primary measurement signal of at least one primary sensor of the primary sensor set from the first measurement and/or a further measurement and (ii) the adjustment. In this way, the primary sensors 110a-f may be calibrated through the secondary sensors 150a-f.

Accordingly, in some embodiments of the system 100, this can be based on the first measurement from each primary sensor 110a-f used to determine the adjustment. The adjustment can be applied to the first measurement at least. This can be a correction applied to the measurement signal (e.g., the raw data) or the property once determined, or any value derived from these. The output can be a corrected or final determination for the property as determined by each of the primary sensors 110a-f.

In alternative or additional embodiments, a further measurement or further measurements may be used or obtained. These can be previous measurements already obtained or may be future measurements to which the adjustment is to be applied. In these embodiments, the control unit 160 may obtain further measurement signal(s) from the primary sensors 110a-f and apply the adjustment to the measurement signal(s) (e.g., the raw data) or the property once determined, or any value derived from these. The output can be a corrected or final determination for the property as determined by each of the primary sensors 110a-f.

The above discussions relate to post-acquisition adjustments. In other or additional embodiments, the adjustment may be used in the process of obtaining the measurement signal. That is, for some sensor types, the adjustment may be used to alter the process by which the control unit 160 obtains the measurement signal so as to compensate for errors or inaccuracies in the previous measurements. For example, if the primary sensors 110a-f are optical sensors, the properties of any light used in the measurement may be adjusted. This may avoid the need to any post-acquisition adjustments.

Other implementations of the calibration operation may be used, for example in system 100. For example, the calibration operation may use plural first measurements and/or plural second measurements to obtain the adjustment.

The above discussion sets out the basic operation of system 100. It will be appreciated that there are numerous parameters in here which can be decided on based on the specific implementation. For example, the frequency with which the selective interaction for the secondary sensor set 152 occurs at may be predetermined, for example so that it is coordinated with the frequency or rate at which the primary sensor set 112 measurements occur. For example, the calibration unit 190 may operate occur each time a measurement is taken by an individual sensor within the primary and/or sensor set, each time a measurement is taken by all of the sensors within the primary and/or secondary sensor set or at selected time intervals. Alternatively, the frequency with which the test fluid selectively interacts with the secondary sensor set 152 may be determined by the degree of adjustment that is required within the system. In some embodiments, if a high degree of adjustment is required, the frequency that the selective interaction of the secondary sensor set 152 with the test fluid may be increase. Such dynamic adjustment of the calibration fluid release frequency may contribute to an increase in accuracy of the readings.

There are several different options for the operation of the fluid monitoring system which will be appreciated by the skilled reader. The examples described are merely for illustrative purposes.

Although in the above embodiment, the main fluid flow path 120 is depicted with first conduit 120a, second conduit 120a, connectors 115a, 115b and primary sensor housing 145, it will be appreciated that other configurations are possible. For example, in some embodiments of the system there is no need for a specific connection to the conduit 115a 115b or the connectors may take any form. In other embodiments, the primary sensor set 112 may be provided within a continuous conduit (i.e. without a separate housing or connectors). In some embodiments, the connectors 115a, 115b are adjustable such that the primary sensor housing 145 can be attached to a variety of different diameters of conduit 120a, 120b. In embodiments where the primary sensor housing 145 is removable this may allow a user to clean or replace the primary sensor set during maintenance.

The second fluid flow path 154 in other embodiments may either be present within the flow path of test fluid, for example, before fluid has flowed through the primary sensor housing 145 or after the fluid has flowed through the primary sensor housing 145.

Although in the above embodiment, a valve is disclosed as providing control over the selective interaction of the secondary sensor set 152 with the test fluid, other configurations are possible. For example, in some embodiments, the selective interaction of the secondary sensor set 152 refers to the calibration unit 190 set being placed in the path of the main fluid flow path 120, for example, in one of the conduits 120a, 120b.

In some embodiments, after the test fluid passes through the secondary sensor set 152, the fluid passes through the waste conduit 182 and the fluid travels to a waste chamber 180. In some embodiments, the waste chamber 180 may be separable from the calibration unit 190. In other or additional embodiments, the waste conduit 182 is connected to a further reservoir or simply connects to a drain, without the need for a waste chamber 180.

In some embodiments, the calibration unit 190 and optionally also the second fluid flow path 154 may be releasably engageable from the main fluid flow path 120 and the primary sensor set 112. This may have the advantage of allowing for the calibration unit 190 to be replaced, but without the need for the primary sensor set 110a-f to be disconnected.

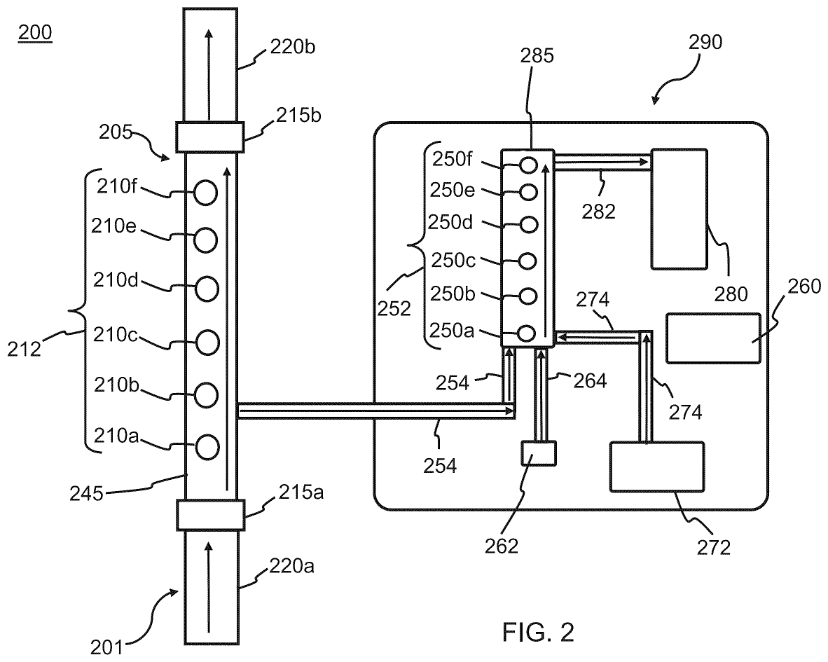

FIG. 2 schematically depicts a system 200 for determining a property of a test fluid according to another embodiment. The system 200 is the same as system 100 except that it includes a second calibration fluid reservoir 270 and associated fluidics.

Accordingly, FIG. 2 depicts a fluid monitoring system 200 for determining a property of the test fluid, comprising a fluid circulation device 201, a sensor assembly 205 and a calibration unit 290. The sensor assembly 205 comprises a primary sensor set 212 which, in this embodiment, comprises six primary sensors 210a-f. As with system 100 of FIG. 1, each of the primary sensors 210a-f is configured to provide a measurement signal corresponding to a different property of test fluid. The sensor assembly 205 also comprises an elongate primary sensor housing 245 in which the primary sensors 210a-210f are located and arranged in a row.

The fluid circulation device 201 defines a main fluid flow path 220. As with system 100, in this system 200, the fluid circulation device 201 comprises a first conduit 220a which connects to an inlet of the primary sensor housing 245 via a connector 215a and a second conduit 220b which connects to an outlet of the primary sensor housing 245 via a connector 215b.

The calibration unit 290 comprises a calibration unit housing 255 which is releasably engageable with the fluid circulation device 201 and in which the components of the calibration unit 290 are located. The calibration unit 290 comprises secondary sensor set 252 in this embodiment in this embodiment comprises six secondary sensors 250a-f. Each of the secondary sensors 250a-f is configured to provide a measurement signal corresponding to a different property of test fluid. The calibration unit 290 also comprises a secondary sensor conduit 285 in which the secondary sensors 250a-f are located and arranged in a row. The calibration unit 290 is fluidly connected to the main fluid flow path 220 by a sampling conduit 254 which provides a secondary fluid flow path. In particular, the sampling conduit 254 extends from an opening in the primary sensor housing 245 to an inlet of the secondary sensor conduit 285 to fluidly connect the main fluid flow path 220 to the secondary sensor set 252. A valve (not depicted) controls the flow of test fluid from the main fluid flow path 220 into the secondary fluid flow path 254.

The calibration unit 290 further comprises a first calibration fluid reservoir 262 comprising a calibration fluid that can be used to calibrate the secondary sensor set 252. The first calibration fluid reservoir 262 is connected to the inlet of the secondary sensor conduit 285 via a first calibration flow path 264. The fluid communication is selective via a valve (not shown) so as to selectively provide calibration fluid to the secondary sensor set 252.

In the system 200 depicted in FIG. 2, there is a second calibration fluid reservoir 272 which is connected to the inlet of the secondary sensor conduit 285 via a second calibration fluid flow path 274. The fluid communication is selective via a valve (not shown) so as to selectively provide calibration fluid to the secondary sensor set 252.

Provision of a secondary calibration fluid which may be utilised by the secondary sensor set 252 is advantageous as it may allow for selection of an appropriate calibration fluid based on the conditions, for example based on the measured properties of the fluid. For example, if there are two calibration fluids, one with an Na⁺ concentration of 0.1 mol/L and another with an Na⁺ concentration of 0.2 mol/L, and a primary sensor 210a-f and/or secondary sensor 250a-f indicate that the concentration of Na⁺ ions in solution is around 0.1 mol/L, the calibration fluid with 0.1 mol/L may be used since this will calibrate the appropriate sensors within the secondary sensor set 252 around this value. The control unit 260 may therefore determine which calibration fluid should be used based on a measurement and dispense one of the calibration fluids to the secondary sensor set 252 accordingly.

In one embodiment, the two calibration fluids may have different time periods for which they are released into the secondary sensor set 252.

The remainder of system 200 is the same as for system 100, including operation. System 200 includes a local control unit 260 which controls operation of the system 200 and calibration of the primary sensor set 212 using the secondary sensor set 252.

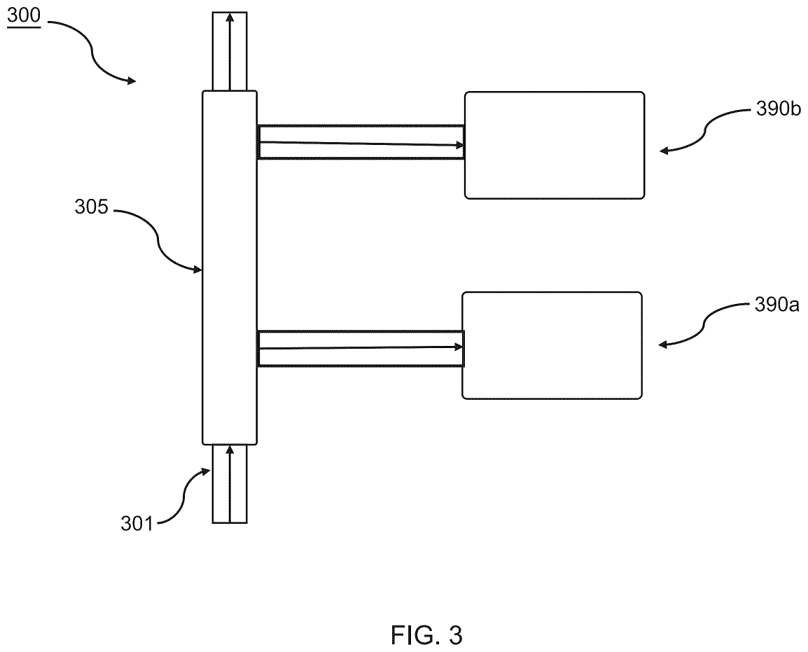

FIG. 3 depicts a system 300 for determining a property of a test fluid. The system 300 is identical to system 100 of FIG. 1 in that it comprises a fluid circulation device 301 which is the same as that of the system 100 of FIG. 1 and a sensor assembly 305 which is the same of that of the system 100 of FIG. 1. The system 300 of FIG. 3 also comprises a calibration unit 390a which is connected to the sensor assembly 305 by a secondary flow path, the calibration unit 390a being identical to that of the system 100 of FIG. 1.

In addition, the system 300 of FIG. 3 further comprises an additional calibration unit 390b which is connected to the sensor assembly 305 by a tertiary flow path, the additional calibration unit 390b being identical to the calibration unit 190 of the system 100 of FIG. 1 and which operates in the same way. The use of an additional calibration unit 390b means that there are second and third sensor sets which can be used in a calibration operation. This allows separate adjustments to be determined or for additional information to be used in determining the adjustment. This may have the advantage of allowing for more than one calibration of the Na⁺ concentration for example. Accordingly, for critical applications, measurements of high precision and reliability may be made. Furthermore, sensors of a different modality may be used within the secondary and third sensor sets such that the sensors may not be prone to the same types of error or drift. Furthermore, this setup may be advantageous since the one sensor set may be replaced while the other sensor set is still operative. This may be advantageous since it allows for calibrated measurements of the primary sensor set to be made without any period in which the measurements cannot be calibrated against any calibration unit sensor set.

Of course, modifications may be present and the calibration unit 390a and additional calibration unit 390b need not be identical to each other or to the calibration units of the abovementioned systems 100, 200. For example, a single control unit may be present, either in one of the calibration unit 390a or additional calibration unit 390b or elsewhere in the system 300.

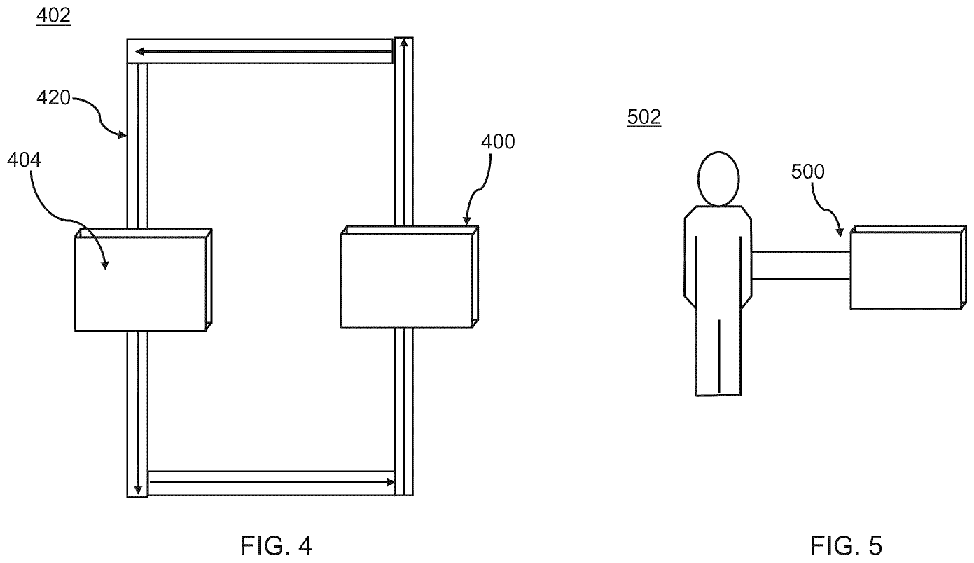

FIG. 4 depicts a fluid flow system 402 with a main fluid pathway 420 having a test fluid flowing through it with the direction as indicated by the arrow. In this embodiment, the test fluid flows through the fluid monitoring system 400, which may correspond to any of the fluid monitoring systems 100, 200, 300 set out in respect of FIGS. 1-3 100. Accordingly, within the fluid monitoring system 400, all measurements and fluid monitoring takes place. The fluid then flows, as indicated by the anticlockwise arrows in FIG. 4 to a fluid process system 404. This step indicates a generic fluid process. Examples of this include a water treatment circuit, a process plant with gas or liquid that is being processed as part of a complete circuit, or any other fluid system. The fluid process system 404 provides the fluid with adequate flow such that it is able to complete a full circuit through the fluid monitoring system 400. The fluid flow system 402 may have the advantage of facilitating continuous measurement of a fluid within a closed system. Accordingly, changes to the measured parameters of a fluid may be monitored over time. In operation, an operator or a processor may make adjustments to the fluid process system 404 based on the output of the fluid monitoring system 400.

In some embodiments of this system, the fluid may be a bodily fluid (such as blood) or water or sewage for treatment. In some embodiments, the fluid is a gas or liquid that is part of an industrial process. In such cases, additional components may be added to the system in order to regulate the flow of fluid around the system.

FIG. 5 depicts an extracorporeal bodily fluid system 502 which connects to a patient so that blood can be taken from the patient and passed through extracorporeal bodily fluid system 502. The properties of the blood can be monitored in the system 502 using a fluid monitoring system 500. The fluid monitoring system 500 may be the fluid monitoring system 100, 200, 300 as described in FIGS. 1 to 3. Accordingly, within this embodiment one or more measurements pertaining to a patient's blood which are calibrated may be made. The information within the extracorporeal bodily fluid system may be used to inform an operator within the system regarding a course of treatment for a patient.

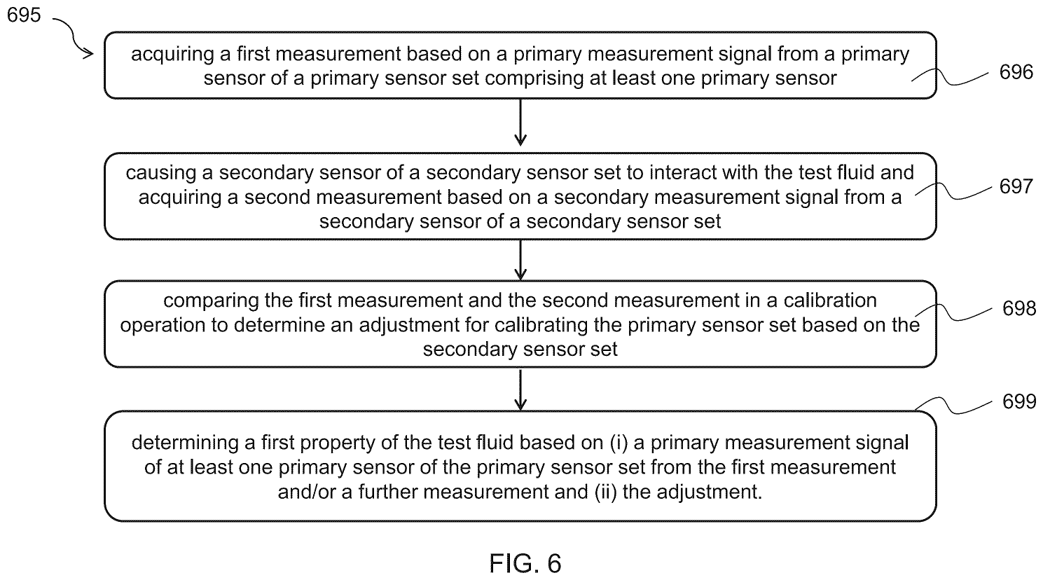

FIG. 6 depicts a method 695 of determining a property of a test fluid provided in a main fluid flow path of a fluid monitoring system, comprising:
acquiring 696 a first measurement based on a primary measurement signal from a primary sensor of a primary sensor set comprising at least one primary sensor, wherein the primary measurement signal is indicative of a first property of a test fluid flowing through a main fluid flow path;
causing 697 a secondary sensor of a secondary sensor set to interact with the test fluid and acquiring a second measurement based on a secondary measurement signal from a secondary sensor of a secondary sensor set, wherein the secondary measurement signal is indicative of the first property of the test fluid;
comparing 698 the first measurement and the second measurement in a calibration operation to determine an adjustment for calibrating the primary sensor set based on the secondary sensor set; and
determining a first property 699 of the test fluid based on (i) a primary measurement signal of at least one primary sensor of the primary sensor set from the first measurement and/or a further measurement and (ii) the adjustment.

It should be understood that the detailed description and specific examples, while indicating exemplary embodiments of the apparatus, systems and methods, are intended for purposes of illustration only and are not intended to limit the scope. These and other features, aspects, and advantages of the apparatus, systems and methods of the present disclosure can be better understood from the description, appended claims or aspects, and accompanying drawings. It should be understood that the Figures are merely schematic and are not drawn to scale. It should also be understood that the same reference numerals are used throughout the figures to indicate the same or similar parts.

Other variations to the disclosed embodiments can be understood and effected by those skilled in the art in practicing the disclosure, from a study of the drawings, the disclosure, and the appended aspects or claims. In the aspects or claims, the word “comprising” does not exclude other elements or steps, and the indefinite article “a” or “an” does not exclude a plurality. The mere fact that certain measures are recited in mutually different dependent aspects or claims does not indicate that a combination of these measures cannot be used to advantage. Any reference signs in the claims should not be construed as limiting the scope.

Aspects of the disclosure will now be set out:

Aspect 1: A fluid monitoring system for determining a property of a test fluid, the fluid monitoring system comprising:
a main fluid flow path through which a test fluid can flow;
a primary sensor set comprising at least one primary sensor configured to provide a primary-measurement signal indicative of a first property of a test fluid flowing through the main fluid flow path;
a secondary sensor set for use in calibrating the primary sensor set, the secondary sensor set comprising a secondary sensor configured to provide a secondary measurement signal indicative of the first property of the test fluid, wherein the system is configured such that the secondary sensor set can selectively interact with test fluid; and
a control unit configured to:
   acquire a first measurement based on the primary measurement signal of the at least one primary sensor and acquire a second measurement based on the secondary measurement signal of the secondary sensor;
   compare the first measurement and the second measurement in a calibration operation to determine an adjustment for calibrating the primary sensor set based on the secondary sensor set; and
   determine a first property of the test fluid based on (i) a primary measurement signal of at least one primary sensor of the primary sensor set from the first measurement and/or a further measurement and (ii) the adjustment.

Aspect 2: The fluid monitoring system of aspect 1, wherein the control unit is configured such that the primary measurement signal used in determining the first property is the primary measurement signal of the first measurement, and wherein determining the first property comprises applying the adjustment to the first measurement.

Aspect 3: The fluid monitoring system of aspect 1 or aspect 2, wherein the control unit is configured such that the primary measurement signal used in determining the first property is a primary measurement signal obtained in a further measurement, and wherein the adjustment is used in obtaining the primary measurement signal in the further measurement.

Aspect 4: The fluid monitoring system of any preceding aspect, further comprising a secondary fluid flow path arranged to selectively receive a portion of the test fluid from the main fluid flow path, wherein the secondary sensor is configured to detect the first property of the test fluid when provided in the second fluid flow path.

Aspect 5: The fluid monitoring system of aspect 4, further comprising a calibration unit, the calibration unit comprising the secondary sensor set and the second fluid flow path, wherein the calibration unit is releasably engageable with the main fluid flow path.

Aspect 6: The fluid monitoring system of any preceding aspect, further comprising a calibration fluid reservoir for receiving a calibration fluid, wherein the system is configured so that the calibration fluid reservoir is in selective fluid communication with secondary sensor set so as to selectively provide calibration fluid to the secondary sensor set.

Aspect 7: The fluid monitoring system of any preceding aspect, wherein the control unit is configured to obtain a plurality of measurements based on the primary measurement signal; and wherein the control unit is configured determine a first property of the test fluid for each of the plurality of measurements based on the primary measurement signal for each measurement and the adjustment.

Aspect 8: The fluid monitoring system of any preceding aspect, wherein the primary sensor set comprises at least one further primary sensor configured to provide a further primary-measurement signal indicative of a further property of a test fluid flowing through the main fluid flow path; and wherein the secondary sensor set further comprises at least one further secondary sensor, each further secondary sensor being configured to provide a further secondary measurement signal indicative of a corresponding further property of the test fluid.

Aspect 9: The fluid monitoring system of any preceding aspect, wherein the primary sensor has a first sensing modality and the secondary sensor has a second sensing modality.

Aspect 10: The fluid monitoring system of aspect 9, wherein the primary sensor comprises an optical sensor and the secondary sensor comprises an electrode for electrochemically sensing the first property.

Aspect 11: An extracorporeal bodily fluid system comprising a circulatory fluid flow path, comprising the fluid monitoring system of any preceding aspect, wherein the main fluid flow path is a circulatory fluid flow path for a bodily fluid.

Aspect 12: A method of determining a property of a test fluid provided in a main fluid flow path of a fluid monitoring system, comprising:
acquiring a first measurement based on a primary measurement signal from a primary sensor of a primary sensor set comprising at least one primary sensor, wherein the primary measurement signal is indicative of a first property of a test fluid flowing through a main fluid flow path;
causing a secondary sensor of a secondary sensor set to interact with the test fluid and acquiring a second measurement based on a secondary measurement signal from a secondary sensor of a secondary sensor set, wherein the secondary measurement signal is indicative of the first property of the test fluid;
comparing the first measurement and the second measurement in a calibration operation to determine an adjustment for calibrating the primary sensor set based on the secondary sensor set; and
determining a first property of the test fluid based on (i) a primary measurement signal of at least one primary sensor of the primary sensor set from the first measurement and/or a further measurement and (ii) the adjustment.

Aspect 13: The method of aspect 12, wherein the step of determining the first property is based on the primary measurement signal of the first measurement and comprises applying the adjustment to the first measurement.

Aspect 14: The method of aspect 12 or aspect 13, wherein the step of determining the first property is based on a primary measurement signal obtained in a further measurement, and wherein the adjustment is used in obtaining the primary measurement signal in the further measurement.

Aspect 15: The method of any one of aspects 12 to 14, further comprising providing a secondary fluid flow path arranged to selectively receive a portion of the test fluid from the main fluid flow path,
wherein causing a secondary sensor of the secondary sensor set to interact with the test fluid comprises providing a portion of the test fluid from the main fluid flow path into the secondary fluid flow path; and
acquiring the second measurement based on a secondary measurement signal from the test fluid when the test fluid is provided in the second fluid flow path.

Aspect 16: The method of any one of aspects 12 to 15, further comprising, prior to acquiring the second measurement, calibrating the secondary sensor of the secondary sensor set.

Aspect 17: The method of any one of aspects 12 to 16, further comprising obtaining a plurality of measurements based on the primary measurement signal,
wherein determining a first property of the test fluid comprises determining a first property for each of the plurality of measurements based on the primary measurement signal for each measurement and the adjustment.

Aspect 18: The method of any one of aspects 12 to 17, further comprising providing a primary sensor having a first sensing modality and providing a secondary sensor having a second sensing modality.

Aspect 19: The method of any one of aspects 12 to 18, further comprising:
providing an extracorporeal bodily fluid system comprising a circulatory fluid flow path for a bodily fluid defining the main fluid flow path; and
providing a bodily fluid to the circulatory fluid flow path, wherein the bodily fluid is the test fluid.

Aspect 20: A computer program comprising computer program code configured, when said computer program is run on one or more physical computing devices, to cause said one or more physical computing devices to implement the method according to any one of aspects 12 to 19.

Aspect 21: One or more non-transitory computer readable media having a computer program stored thereon, the computer program comprising computer program code which is configured, when said computer program is run on one or more physical computing devices, to cause one or more physical computing devices to implement the method according to any one of aspects 12 to 19.

1. A fluid monitoring system for determining a property of a test fluid, the fluid monitoring system comprising:
a main fluid flow path through which a test fluid can flow;
a primary sensor set comprising at least one primary sensor configured to provide a primary-measurement signal indicative of a first property of a test fluid flowing through the main fluid flow path;
a secondary sensor set for use in calibrating the primary sensor set, the secondary sensor set comprising a secondary sensor configured to provide a secondary measurement signal indicative of the first property of the test fluid, wherein the system is configured such that the secondary sensor set can selectively interact with test fluid; and
a control unit configured to:
acquire a first measurement based on the primary measurement signal of the at least one primary sensor and acquire a second measurement based on the secondary measurement signal of the secondary sensor;
compare the first measurement and the second measurement in a calibration operation to determine an adjustment for calibrating the primary sensor set based on the secondary sensor set; and
determine a first property of the test fluid based on (i) a primary measurement signal of at least one primary sensor of the primary sensor set from the first measurement and/or a further measurement and (ii) the adjustment.

2. The fluid monitoring system of claim 1, wherein the control unit is configured such that the primary measurement signal used in determining the first property is the primary measurement signal of the first measurement, and wherein determining the first property comprises applying the adjustment to the first measurement.

3. The fluid monitoring system of claim 1, wherein the control unit is configured such that the primary measurement signal used in determining the first property is a primary measurement signal obtained in a further measurement, and wherein the adjustment is used in obtaining the primary measurement signal in the further measurement.

4. The fluid monitoring system of claim 1, further comprising a secondary fluid flow path arranged to selectively receive a portion of the test fluid from the main fluid flow path, wherein the secondary sensor is configured to detect the first property of the test fluid when provided in the second fluid flow path.

5. The fluid monitoring system of claim 4, further comprising a calibration unit, the calibration unit comprising the secondary sensor set and the second fluid flow path, wherein the calibration unit is releasably engageable with the main fluid flow path.

6. The fluid monitoring system of claim 1, wherein the control unit is configured to obtain a plurality of measurements based on the primary measurement signal; and wherein the control unit is configured determine a first property of the test fluid for each of the plurality of measurements based on the primary measurement signal for each measurement and the adjustment.

7. The fluid monitoring system of claim 1, wherein the primary sensor set comprises at least one further primary sensor configured to provide a further primary-measurement signal indicative of a further property of a test fluid flowing through the main fluid flow path; and wherein the secondary sensor set further comprises at least one further secondary sensor, each further secondary sensor being configured to provide a further secondary measurement signal indicative of a corresponding further property of the test fluid.

8. The fluid monitoring system of claim 1, wherein the primary sensor has a first sensing modality and the secondary sensor has a second sensing modality.

9. The fluid monitoring system of claim 8, wherein the primary sensor comprises an optical sensor and the secondary sensor comprises an electrode for electrochemically sensing the first property.

10. An extracorporeal bodily fluid system comprising a circulatory fluid flow path, comprising the fluid monitoring system of claim 1, wherein the main fluid flow path is a circulatory fluid flow path for a bodily fluid.

11. A method of determining a property of a test fluid provided in a main fluid flow path of a fluid monitoring system, comprising:
acquiring a first measurement based on a primary measurement signal from a primary sensor of a primary sensor set comprising at least one primary sensor, wherein the primary measurement signal is indicative of a first property of a test fluid flowing through a main fluid flow path;
causing a secondary sensor of a secondary sensor set to interact with the test fluid and acquiring a second measurement based on a secondary measurement signal from a secondary sensor of a secondary sensor set, wherein the secondary measurement signal is indicative of the first property of the test fluid;
comparing the first measurement and the second measurement in a calibration operation to determine an adjustment for calibrating the primary sensor set based on the secondary sensor set; and
determining a first property of the test fluid based on (i) a primary measurement signal of at least one primary sensor of the primary sensor set from the first measurement and/or a further measurement and (ii) the adjustment.

12. The method of claim 11, wherein the determining the first property is based on the primary measurement signal of the first measurement and comprises applying the adjustment to the first measurement.

13. The method of claim 11, wherein the determining the first property is based on a primary measurement signal obtained in a further measurement, and wherein the adjustment is used in obtaining the primary measurement signal in the further measurement.

14. The method of claim 11, further comprising providing a secondary fluid flow path arranged to selectively receive a portion of the test fluid from the main fluid flow path,
wherein causing a secondary sensor of the secondary sensor set to interact with the test fluid comprises providing a portion of the test fluid from the main fluid flow path into the secondary fluid flow path; and
acquiring the second measurement based on a secondary measurement signal from the test fluid when the test fluid is provided in the second fluid flow path.

15. The method of claim 11, further comprising, prior to acquiring the second measurement, calibrating the secondary sensor of the secondary sensor set.

16. The method of claim 11, further comprising obtaining a plurality of measurements based on the primary measurement signal,
wherein determining a first property of the test fluid comprises determining a first property for each of the plurality of measurements based on the primary measurement signal for each measurement and the adjustment.

17. The method of claim 11, further comprising providing a primary sensor having a first sensing modality and providing a secondary sensor having a second sensing modality.

18. The method of claim 11, further comprising:
providing an extracorporeal bodily fluid system comprising a circulatory fluid flow path for a bodily fluid defining the main fluid flow path; and
providing a bodily fluid to the circulatory fluid flow path, wherein the bodily fluid is the test fluid.

19. A computer program comprising computer program code configured, when said computer program is run on one or more physical computing devices, to cause said one or more physical computing devices to implement the method according to claim 11.

20. One or more non-transitory computer readable media having a computer program stored thereon, the computer program comprising computer program code which is configured, when said computer program is run on one or more physical computing devices, to cause one or more physical computing devices to implement the method according to claim 11.
